(19) United States
(12) Patent Application Publication
(10) Pub. No.: US 2021/0386419 A1
(43) Pub. Date: Dec. 16, 2021

(54) POSITIONING DEVICES FOR ABNORMAL TISSUES

(71) Applicant: National Taiwan University, Taipei (TW)

(72) Inventors: Tai-Horng YOUNG, Taipei (TW); Ke-Cheng CHEN, Taipei (TW); Jin-Shing CHEN, Taipei (TW); You-Ren JI, Taipei (TW); Ting-Yu TSAI, Taipei (TW); Yu-Chun CHOU, Taipei (TW)

(21) Appl. No.: 17/325,476

(22) Filed: May 20, 2021

(30) Foreign Application Priority Data
Jun. 14, 2020 (TW) 109119984

(51) Int. Cl. A61B 17/04

(52) U.S. Cl. CPC A61B 17/0401; A61B 2017/0409

(57) ABSTRACT

A positioning device for positioning an abnormal tissue is provided. The positioning device comprises a positioning tube and a positioning suture accommodated in the positioning tube. When the positioning suture is injected into the abnormal tissue, a head portion and a portion of a body portion of the positioning suture are retained inside the abnormal tissue while the rest of the body portion being exposed outside the abnormal tissue, thereby marking a specific area accordingly to facilitate the subsequent excision process.

### CROSS REFERENCE TO RELATED APPLICATION

This application claims the benefit of Taiwan Patent Application No. 109119984, filed on Jun. 14, 2020. The entirety of the Application is incorporated herein by reference.

### BACKGROUND OF THE INVENTION

### 1. Field of the Invention

The present invention relates to a positioning device, particularly a positioning device for positioning abnormal tissues.

### 2. Description of Related Art

Positioning is a common procedure required in medical practices. For example, in a tumor excision surgery, a positioning procedure is usually required to target the lesion using computerized tomographic guidance with localization techniques before an excision procedure. However, the dye used for the positioning procedure, although intuitive, is time-sensitive so that the excision procedure must be performed as soon as possible after the positioning procedure, otherwise, the dye may diffuse and mislead the evaluation because of the difficulty in controlling the staining area. Also, the other drawback of the dye is that it may trigger allergic reactions in some patients. In addition to said dye-based positioning, another approach commonly used is “metal implantation”, in which a spring ring or a small needle (hookwire) is implanted around the lesion for positioning, but this method also has the risk of implant displacement and bleeding.

Accordingly, researchers in the field are now developing new methods for positioning to reduce the risk and improve the accuracy of the learned positioning procedure.

### SUMMARY OF THE INVENTION

A positioning device for medical procedures is disclosed to improve the drawbacks of known positioning procedures. The positioning device not only provides good fixation and ensures precise positioning, but also reduces invasive injuries during the positioning procedure and prolongs the operable time between the positioning procedure and excision procedure.

The positioning device for abnormal tissues of the present invention comprises a positioning tube, a guiding needle, and a positioning suture. The positioning tube is provided with a receiving space and has an outer diameter. The guiding needle has an inner diameter that is greater than the outer diameter of the positioning tube so that the guiding needle is able to be disposed on an outer side of the positioning tube. The positioning suture has a body portion and a head portion connected to the body portion, in which the widest part of the head portion corresponds to the inner diameter of the guiding needle to allow the positioning suture to be accommodated in the receiving space. Therefore, when the guiding needle is inserted into the abnormal tissue, the positioning tube is inserted into the abnormal tissue along with the inner diameter of the guiding needle; and when the positioning suture is injected into the abnormal tissue, the head portion and a portion of the body portion are retained inside the abnormal tissue while the rest of the body portion being exposed outside the abnormal tissue.

According to an embodiment, the positioning tube has an opening, whereby the head portion of the positioning suture is at least partially exposed at the opening while the positioning suture is accommodated in the receiving space.

According to an embodiment, the head portion is provided with a top end and a tail end correspondingly, and the top end is a closed end.

According to an embodiment, the widest part of the head portion is located at the opening with the top end of the head portion being exposed outside the opening when the positioning suture is accommodated in the receiving space.

According to an embodiment, the positioning device further comprises a syringe. The syringe is sleeved on the positioning tube via an end opposite to the opening to provide a pushing force. Through the pushing force, the positioning suture accommodated in the receiving space can be pushed out to complete the positioning.

According to an embodiment, the opening is beveled, and the body portion and the head portion are integrally formed. However, in other embodiments, the material used to form the body portion and the head portion may not be the same, or the body portion and the head portion may be individually manufactured and then assembled. The present invention is not limited thereto.

According to an embodiment, the receiving space is further filled with a biologically acceptable substance. The biologically acceptable substance may be selected as required without any limitation. For example, the biologically acceptable substance may be a bio-gel to aid in wound healing, a drug to inhibit the growth of the lesion, or a saline solution to avoid introducing air into the body and has a lubricating effect to assist movements of the positioning suture during the procedure. According to an embodiment, a surface of the positioning suture is coated with a developer. For example, developers such as Indocyanine Green (ICG), iodine oil, methylene blue, and the like, can be used.

According to an embodiment, the head portion is sleeved on the body portion, and the positioning suture further comprises a plurality of first fixing portions which are fixed to the head portion on the body portion.

According to an embodiment, the body portion further comprises a plurality of second fixing portions that are distributed thereon in a range of 15 cm from the head portion.

According to an embodiment, the second fixing portion is formed in a configuration of barb-shaped, fish bone-shaped, hollow cone-shaped, knot-shaped, or comb-shaped.

According to an embodiment, the head portion has elasticity. For example, the head portion may be made of a material having elasticity, whereby the head portion is suitably flexible to allow it to be accommodated in the guiding needle.

According to an embodiment, the head portion is in the form of cone-shaped or bullet-shaped.

According to an embodiment, the head portion is in the form of umbrella-shaped, knot-shaped, hollow cone-shaped, or comb-shaped.

The above and other features and advantages of the present invention will be clearly understood by the following description of the embodiments.

### BRIEF DESCRIPTION OF THE DRAWINGS

FIG. 1 is a perspective schematic view showing a positioning device of an embodiment of the present invention;

FIG. 2 is a perspective schematic view showing a positioning device provided with a syringe of an embodiment of the present invention;

FIG. 3 is a perspective schematic view showing a positioning device of another embodiment of the present invention;

FIG. 4A to FIG. 4D are perspective schematic views showing the head portions of other embodiments;

FIG. 5 is a schematic diagram showing a configuration of a positioning suture of an embodiment of the invention;

FIG. 6 is a schematic diagram showing a configuration of a positioning suture of another embodiment of the invention;

FIG. 7 is a schematic diagram showing a configuration of a positioning suture of another embodiment of the invention; and

FIG. 8 to FIG. 10 are perspective schematic views showing positioning sutures and the second fixing portions of other embodiments.

### DETAILED DESCRIPTION OF THE PREFERRED EMBODIMENT

Hereinafter, an example is provided to illustrate the embodiment of the present invention. The advantages and efficacy of the invention will become more apparent by the disclosure of the invention. The accompanying drawings are simplified and used for illustrative purposes. The number, shape, and size of the components shown in the drawings may be modified to suit the actual situation, and the configuration of the components may be more complex as well. Other aspects of the invention may also be practiced or applied, and variations and adjustments may be made without departing from the spirit and scope of the invention as defined herein.

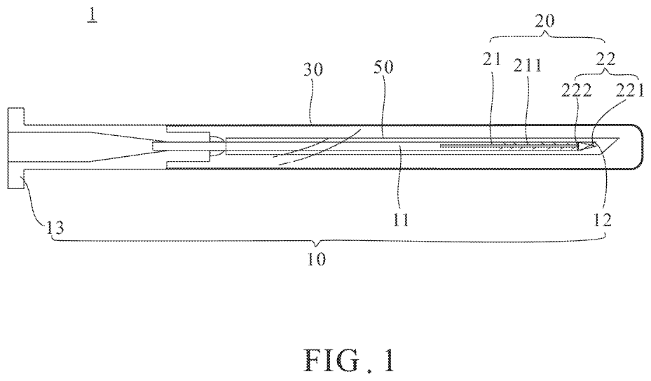

FIG. 1 is a perspective schematic view of an embodiment of the present invention showing a positioning device 1. The positioning device 1 mainly comprises a positioning tube 10 and a positioning suture 20. Considering the safety of the user when using it, the positioning device 1 of the present embodiment may also include a protective cover 30 for covering the positioning tube 10 properly.

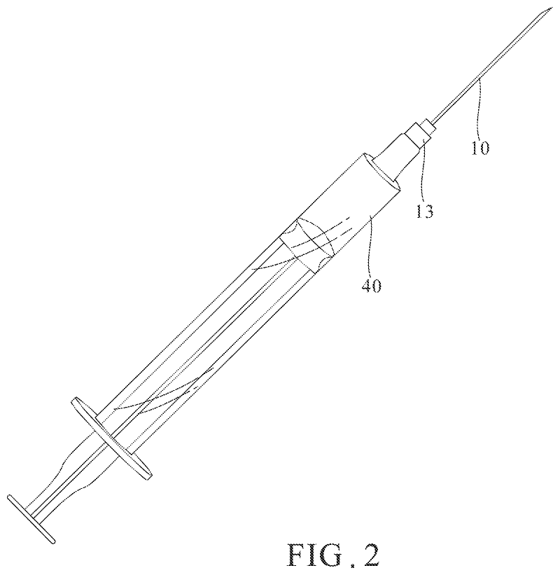

The positioning tube 10 is a metallic hollow tube provided with a receiving space 11. The positioning tube 10 has two opposite ends, one of which is an opening 12, which is beveled; the other end of which is a connecting part 13 corresponding to the opening 12. In another embodiment, the opening 12 may also be realized as a blunt end. Besides, as shown in FIG. 2, the connecting part 13 may then be connected to a syringe 40 to provide a pushing force through the syringe 40 during positioning.

The positioning suture 20 comprises a body portion 21 and a head portion 22 connected to the body portion 21. The body portion 21 and the head portion 22 are integrally formed. The material to be used is not particularly limited, but preferably a material that gives the positioning suture 20 flexibility, such as nylon, degradable or non-degradable polymers, and the like.

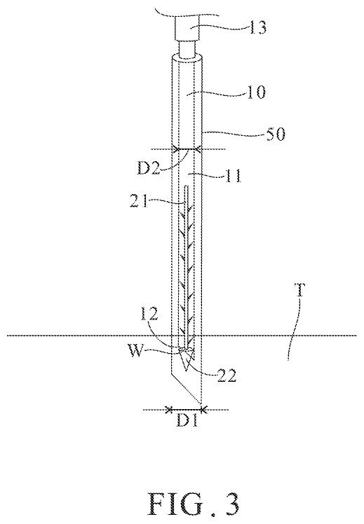

In a preferred embodiment, regarding FIG. 1 and FIG. 3, the positioning device 1 may further include a guiding needle 50 to assist in positioning. The guiding needle 50 is also a metallic hollow tube and has an inner diameter D 1. The inner diameter D1 is larger than the outer diameter D2 of the positioning tube 10 to allow the guiding needle 50 to be disposed on the outer side of the positioning tube 10. Therefore, when the positioning suture 20 is accommodated in the receiving space 11, the head portion 22 of the positioning suture 20 is at least partially exposed outside the opening 12, and preferably, as shown in FIG. 3, the widest part W of the corresponds to the inner diameter D1 of the positioning tube 10 while the widest part W is located at the opening end 12. However, in the embodiment where the opening 12 is a blunt end, the head portion 22 is exposed outside the opening 12 and the widest part W is located closely adjacent to the opening 12.

During implementation, first, inserting the guiding needle 50 into the target abnormal tissue T, and allowing the positioning tube 10 to inserted into the abnormal tissue T along with the inner diameter of the guiding needle 50. Then, injecting the positioning suture 20 into the abnormal tissue, allowing the head portion 22 and a portion of the body portion 21 to be retained inside the abnormal tissue T while the rest of the body portion 21 to be exposed outside the abnormal tissue T.

In the case of excising a lung cancer nodule, first, inserting the guiding needle 50 into the abnormal tissue T at the targeted position for initial positioning. Then, filling the positioning tube 10 with the positioning suture 20, allowing the positioning tube 10 to insert the target position along with the inner diameter of the guiding needle 50. In other embodiments, the guiding needle 50 can be omitted and the positioning tube 10 can be inserted directly into the abnormal tissue T at the target position for initial positioning. Alternatively, it is possible to omit the positioning tube 10 while inserting the guiding needle 50 into the abnormal tissue T at the target position and feeding the positioning suture 20 into the tube of the guiding needle 50 to complete the positioning procedure. Moreover, the positioning can be completed by each of these embodiments simply by the positioning suture 20 without using a syringe 40.

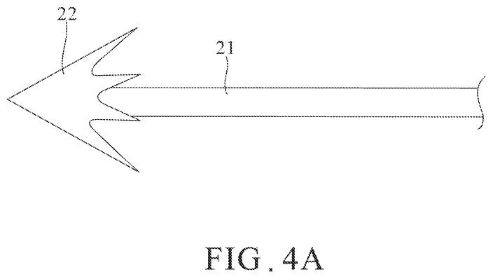

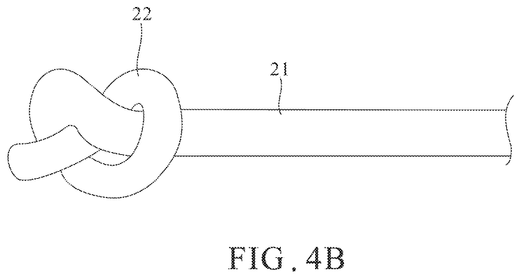

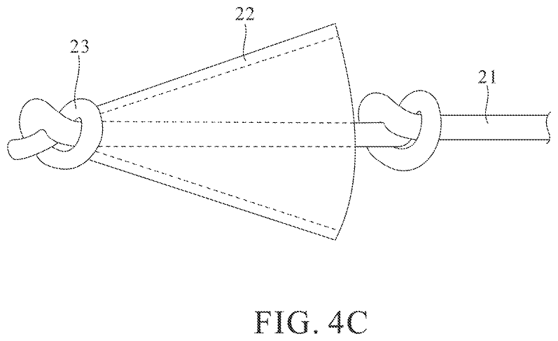

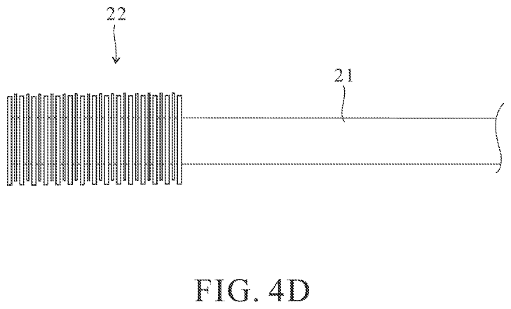

As shown in the embodiments and the drawings above, the head portion 22 is preferably configured with a narrow top end 221 and a wide tail end 222 as a cone-shaped or a bullet-shaped configuration. The head portion 22 may also be configured as an umbrella-shaped structure as shown in FIG. 4A. In the embodiment, the top end 221 is a closed end and the diameter of the tail end 222 at the widest part W of the head portion 22 corresponds to the inner diameter of the receiving space 11, and thus the positioning suture 20 can be accommodated in the receiving space 11 without applying additional external forces. At this time, the top end 221 of the head portion 22 is exposed outside the opening 12.

In other embodiments, the head portion 22 may also be constructed in a configuration ofknot-shaped (FIG. 4B), hollow cone-shaped (FIG. 4C), or comb-shaped (FIG. 4D) structure, and the number of the head portions 22 may be varied and arranged sequentially to enhance the fixation to the abnormal tissue T during implementation. For example, as shown in FIG. 4C, the hollow cone-shaped head portion 22 is movably disposed on the body portion 21 and is fixed to an end of the body portion 21 by a plurality of first fixing portions 23. For example, the first fixing portion 23 may be realized in a spherical or knotted structure to secure the head portion 22 to an end of the body portion 21.

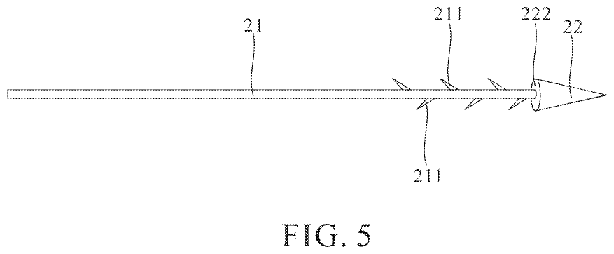

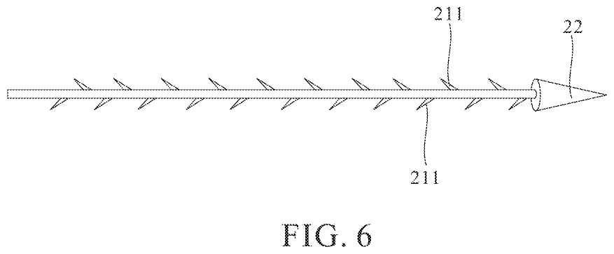

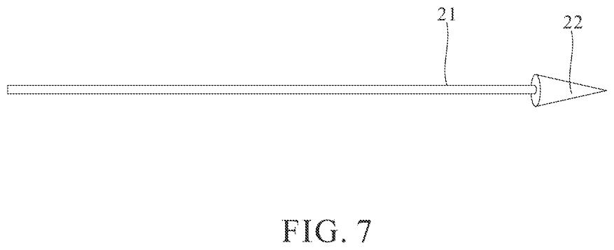

For the positioning suture 20, it is preferred that the leading section of the body portion 21, as illustrated in FIG. 5, is formed with a plurality of second fixing portions 211. The second fixing portions 211 are disposed in an inclined direction toward the body portion 21, namely, each second fixing section 211 is at an angle of less than 90 degrees to the body portion 21. In this way, the body portion 21 can be easily inserted into the abnormal tissue T during the positioning process and is not easily dislodged. Specifically, the part with the second fixing portions 211 may be formed on the leading section of the body portion 21, wherein the leading section is defined as a 15-centimeter-long region started from a point where the tail end 222 of the head portion 22 and the body portion 21 are connected. In this case, the rest of the body portion 21 is not provided with the second fixing portion 211. However, the present invention is not limited thereto. In other embodiments, the positioning suture 20 may have the second fixing portions 211 disposed fully on the body portion 21 as shown in FIG. 6, or the body portion 21 may be a smooth suture without the second fixing portions 211 to avoid damage to the human skin during positioning (FIG. 7).

Following the above, as illustrated in FIG. 5 and FIG. 6, the second fixing portions 211 may be formed in a configuration of barb-shaped or fish bone-shaped (or curved spike). Notably, these second fixing portions 211 can be used to fix the positioning suture 20 in the abnormal tissue T to prevent it from slipping. Therefore, in case these second fixing portions 211 are formed on the body portion 21, the angle and direction between each second fixing portion 211 and the body portion 21 can be different. Hence, even though the positioning suture 20 is pulled by an external force, it can still be fixed to the tissue securely because these second fixing portions 211 are formed with different angles.

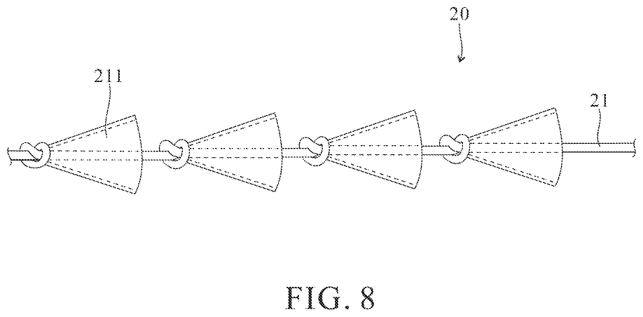

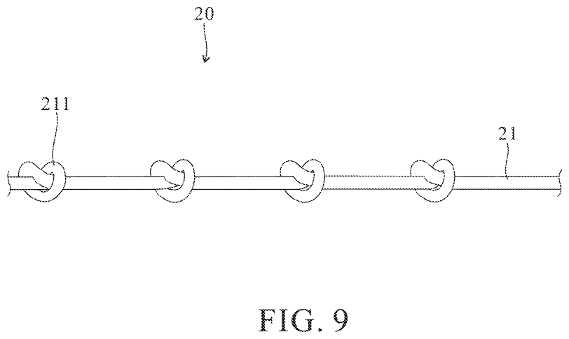

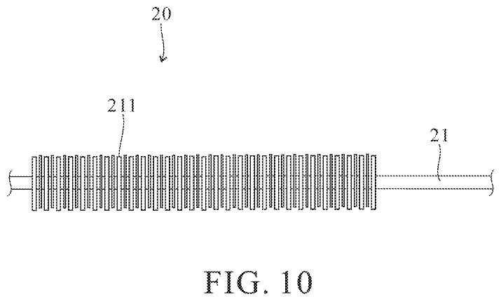

In other embodiments, each second fixing portion 211 can also be realized as a hollow cone-shaped (FIG. 8), a knot-shaped (FIG. 9), or a comb-shaped (FIG. 10). Meanwhile, such as the arrangements, sizes, spacing of the second fixing portion 211 can be adjusted as desired so that the positioning suture 20 can be easily inserted into the abnormal tissue T by pushing. Meanwhile, when the positioning tube 10 and/or the guiding needle 50 is withdrawn, the positioning suture 20 can be retained and fixed in the tissue without being dislodged.

Except for the positioning suture 20, the receiving space 11 of the positioning tube 10 can also be filled with a biologically acceptable substance that can be injected continuously during the positioning process. The substance not only helps to propel the positioning suture 20 but also prevents air from being injected into the body, causing adverse effects such as blood clots. The “biologically acceptable substance” can be selected as needed, for example, a bio-gel that helps the wound to heal, a drug that inhibits the growth of the lesion, or simply a saline solution as a mediator to propel the positioning suture 20.

In the embodiment, the positioning suture 20 may be suitably processed to allow the developer to be attached thereon. There is no limitation on the type of developer. Indocyanine Green (ICG), iodine oil, methylene blue, and other dyes can be used to enhance the positioning effect of the positioning suture 20 of the present invention.

For example, when removing a nodule of lung cancer, the positioning suture 20 and the saline solution can be filled into the positioning tube 10. Then, inserting the positioning tube 10 into the target position and applying an external force to push the syringe 40 to generate a pushing force in the positioning tube 10 for pushing the positioning suture 20 and the saline solution out of the receiving space 11. Then, the positioning suture 20 reached the target position has the head portion 22 and part of the body portion 21 with the second fixing portion 211 retained inside the tissue, while part of the body portion 21 exposed outside the tissue for positioning, thereby marking the area to be removed for subsequent surgical excision.

The positioning suture 20 of the present invention not only provides good fixation and ensures precise positioning, but also includes barbs to reduce the risk of displacement of the positioning suture due to movement of the body or organ before an operation, extending the operable time between the positioning procedure and excision procedure. However, these are just a few examples of applications. The positioning device 1 of the present invention is not limited to be used in a tumor excision surgery but also applicable to various organs and tissues for injection and positioning surgery.

What is claimed is:

1. A positioning device for an abnormal tissue, comprising:
a positioning tube provided with a receiving space and having an outer diameter;
a guiding needle provided with an inner diameter, wherein the inner diameter is greater than the outer diameter of the positioning tube so that the guiding needle is disposed on an outer side of the positioning tube; and
a positioning suture having a body portion and a head portion connected to the body portion, wherein the widest part of the head portion corresponds to the inner diameter of the guiding needle to allow the positioning suture to be accommodated in the receiving space;
wherein, when the guiding needle is inserted into the abnormal tissue, the positioning tube is inserted into the abnormal tissue along with the inner diameter of the guiding needle; and when the positioning suture is injected into the abnormal tissue, the head portion and a portion of the body portion are retained inside the abnormal tissue while the rest of the body portion being exposed outside the abnormal tissue.

2. The positioning device for an abnormal tissue as claimed in claim 1, wherein the positioning tube has an opening, whereby the head portion of the positioning suture is at least partially exposed at the opening while the positioning suture is accommodated in the receiving space.

3. The positioning device for an abnormal tissue as claimed in claim 2, wherein the head portion is provided with a top end and a tail end correspondingly, and the top end is a closed end.

4. The positioning device for an abnormal tissue as claimed in claim 3, wherein the widest part of the head portion is located at the opening with the top end of the head portion being exposed outside the opening when the positioning suture is accommodated in the receiving space.

5. The positioning device for an abnormal tissue as claimed in claim 4, wherein the positioning device for an abnormal tissue further comprises a syringe sleeved on the positioning tube via an end opposite to the opening to provide a pushing force.

6. The positioning device for an abnormal tissue as claimed in claim 2, wherein the opening is beveled.

7. The positioning device for an abnormal tissue as claimed in claim 1, wherein the body portion and the head portion are integrally formed.

8. The positioning device for an abnormal tissue as claimed in claim 1, wherein the receiving space is further filled with a biologically acceptable substance.

9. The positioning device for an abnormal tissue as claimed in claim 1, wherein a surface of the positioning suture is coated with a developer.

10. The positioning device for an abnormal tissue as claimed in claim 1, wherein the head portion is sleeved on the body portion, and the positioning suture further comprises a plurality of first fixing portions to fix the head portion on the body portion.

11. The positioning device for an abnormal tissue as claimed in claim 1, wherein the body portion further has a plurality of second fixing portions distributed thereon in a range of 15 cm from the head portion.

12. The positioning device for an abnormal tissue as claimed in claim 11, wherein each of the second fixing portions is formed in a configuration of barb-shaped, fish bone-shaped, hollow cone-shaped, knot-shaped, or comb-shaped.

13. The positioning device for an abnormal tissue as claimed in claim 1, wherein the head portion has elasticity.

14. The positioning device for an abnormal tissue as claimed in claim 3, wherein the head portion is in a form of cone-shaped or bullet-shaped.

15. The positioning device for an abnormal tissue as claimed in claim 1, wherein the head portion is in a form of umbrella-shaped, knot-shaped, hollow cone-shaped, or comb-shaped.
